SEQUENCE LISTING (12 sequences)

SEQ ID NO: 1 - DNA, 21 bp, Artificial Sequence
  Synthetic forward primer for Cdc42
ctgccaaccatgacaacctaa

SEQ ID NO: 2 - DNA, 21 bp, Artificial Sequence
  Synthetic reverse primer for Cdc42
agacaaaacaacaaggtccag

SEQ ID NO: 3 - DNA, 1128 bp, Mus musculus
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatgttcgaacgaagttcctattctctagaaagtataggaacttcgctagactagtacgcgtgtacaccttgtaattgctgctctgagcaagttgccattttttctttttagaggttttcagtcatagcagtaatgctagttctggtttgagtggctgagcctgttgctaggggaaaaaagtatggatttaaacataaatcaataaaataattgtctttaatttcttcttaggacaagatctaatttgaaatattaaaagtggatacaaaactgtttccgaaatgcagacaattaagtgtgttgttgttggtgatggtgctgttggtaaaacatgtctcctgatatcctacacaacaaacaaattcccatcggaatatgtaccaactgtaagtataaaggctttttactagcaaaagattgtaatgtagtgtctgtccattggaaaacacttggcctgcctgcagtatttttgactgtcttgccctttaaaaaaaattaaattttactacctttattactttgtggggtgtgtgttataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 4 - DNA, 561 bp, Mus musculus
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 5 - DNA, 23 bp, Artificial Sequence
  Synthetic forward primer for Gapdh
aaggtcggtgtgaacggatttgg

SEQ ID NO: 6 - DNA, 23 bp, Artificial Sequence
  Synthetic reverse primer for Gapdh
cgttgaatttgccgtgagtggag

SEQ ID NO: 7 - DNA, 22 bp, Artificial Sequence
  Synthetic forward primer for Col1a1
cctcagggtattgctggacaac

SEQ ID NO: 8 - DNA, 22 bp, Artificial Sequence
  Synthetic reverse primer for Col1a1
cagaaggaccttgtttgccagg

SEQ ID NO: 9 - DNA, 22 bp, Artificial Sequence
  Synthetic forward primer Human Tgfb1
tacctgaacccgtgttgctctc

SEQ ID NO: 10 - DNA, 22 bp, Artificial Sequence
  Synthetic reverse primer for Human Tgfb1
gttgctgaggtatcgccaggaa

SEQ ID NO: 11 - DNA, 22 bp, Artificial Sequence
  Synthetic forward primer for Mouse Tgfb1
tgatacgcctgagtggctgtct

SEQ ID NO: 12 - DNA, 22 bp, Artificial Sequence
  Synthetic reverse primer for Mouse Tgfb1
cacaagagcagtgagcgctgaa

(19) United States
(12) Patent Application Publication
(10) Pub. No.: US 2022/0273822 A1
(43) Pub. Date: Sep. 1, 2022

(54) ANIMAL MODEL OF IDIOPATHIC PULMONARY FIBROSIS, ITS CONSTRUCTION METHOD AND USE

(71) Applicant: National Institute of Biological Sciences, Beijing, Beijing (CN)

(72) Inventors: Nan Tang, Beijing (CN); Huijuan Wu, Beijing (CN)

(73) Assignee: National Institute of Biological Sciences, Beijing, Beijing (CN)

(21) Appl. No.: 17/614,737

(22) PCT Filed: May 30, 2019
(86) PCT No.: PCT/CN2019/089357

§ 371 Date: Nov. 29, 2021

(51) Int. Cl. A61K 49/00; A61P 11/00; A01K 67/027

(52) U.S. Cl. CPC A61K 49/0008; A61P 11/00; A01K 67/0275; A01K 2217/075; A01K 2217/15; A01K 2227/105; A01K 2267/035

(57) ABSTRACT

The present invention relates to a method for constructing an animal model of pulmonary fibrosis, in particular, idiopathic pulmonary fibrosis (IPF), the constructed animal model using the said method, and a method for screening the candidate drugs for treating pulmonary fibrosis, in particular, idiopathic pulmonary fibrosis (IPF).

### PRIORITY CLAIM AND CROSS-REFERENCE

The present application is a National Stage Application, filed under 35 U.S.C. 371, of International Patent Application No. PCT/CN2019/089357, filed on May 30, 2019, which is incorporated herein by reference in its entirety.

### SEQUENCE LISTING

This application contains a Sequence Listing, which is submitted electronically via EFS- Web in ASCII format with a file name H5292-00002-SEQTXT, creation date of Mar. 3, 2022, and a size of 5 kB. This sequence listing submitted is part of the specification and is herein incorporated by reference in its entirety.

Introduction

Fibrosis, the thickening and scarring of connective tissue that can result from injury, is characterized by the excessive proliferation of fibroblast cells and the accumulation of extracellular matrix (ECM) components. This disorder, which is commonly observed in organs including lungs, livers, and kidneys, among many others, causes disrupted tissue architecture and leads to major impairments in organ function^{1,2}. Indeed, fibrosis can develop in nearly every organ and is a major cause of end-stage organ failure and death in a large variety of chronic diseases³. A common feature of pulmonary fibrosis is the excessive proliferation of fibroblasts around the air sacs of lungs (alveoli)⁴. Extensive biomedical studies have established that an increased number of fibroblasts, in combination with their excessive ECM deposition in the lung ultimately cause alveolar structure destruction, decreased lung compliance, and disrupted gas exchange function⁵⁻⁷.

The most common type of pulmonary fibrosis is idiopathic pulmonary fibrosis (IPF). This disorder eventually affects entire lung lobes, but it begins with microscopic fibrotic lesions that occur at the peripheral regions and slowly progress inward, and this fibrosis can ultimately lead to respiratory failure ^{8,9}. IPF is a fatal disease with the median survival time being only 2-4 years from diagnosis¹⁰. The mechanisms and nature of the pathological progression of IPF are not fully understood, although multiple studies have implicated contributions from a specific subset of alveolar epithelial cells-alveolar type II (AT2) cells^{4,11}.

The alveolar epithelia of lungs are composed of a combination of both alveolar type I (AT1) and type II (AT2) cells. AT2 cells are the alveolar stem cells, and can differentiate into AT1 cells during alveolar homeostasis and post-injury repair^{12,13}. AT1 cells-which ultimately constitute fully 95% of the alveolar surface in adult lungs-are large squamous cells that function as the epithelial component of the thin air-blood barrier¹⁴. In IPF tissues, abnormal hyperplastic AT2 cells are typically located adjacent to fibroblastic foci¹⁵, and the gene mutants that affect the functions of AT2 cells are frequently observed in IPF tissues in the clinic^{16,17}. In addition, recent advances in identifying the molecular profiles of IPF lungs showed that TGFβsignaling (a common fibrotic signaling in many fibrotic diseases) is activated in the AT2 cells of IPF lungs¹⁸.

The pulmonary fibrosis patient has decreased lung compliance, disrupted gas exchange, and ultimately respiratory failure and death. It is estimated that IPF affects 1 of 200 adults over the age of 65 in the United States, with a median survival time of 2-4 years. In China, the estimated incidence of IPF is 3-5/100,000, accounting for about 65% of all interstitial lung diseases. The diagnosis is usually made between 50 and 70 years old, and the ratio of male to female is 1.5 to 2:1. The survival time of the patient is usually only 2-5 years.

Currently, there is no medicine for curing IPF. Two known drugs, nintedanib and pirfenidone, have similar effects on the rate of decline in forced vital capacity over 1 year. Although both drugs showed a tendency of reducing mortality, these two drugs failed to show significantly increased survival time. One of main reasons is that there is no ideal animal model of pulmonary fibrosis, in particular, idiopathic pulmonary fibrosis (IPF), so as to screen candidate drugs for treating pulmonary fibrosis, in particular, idiopathic pulmonary fibrosis (IPF).

Summary of the Invention

The present invention relates to a method for constructing an animal model of pulmonary fibrosis, in particular, idiopathic pulmonary fibrosis (IPF), the constructed animal model using the said method, and a method for screening the candidate drugs for treating pulmonary fibrosis, in particular, idiopathic pulmonary fibrosis (IPF). The present invention provides a constructed disease animal model of pulmonary fibrosis, in particular, idiopathic pulmonary fibrosis (IPF), and the constructed animal model may be used to study pulmonary fibrosis, in particular, idiopathic pulmonary fibrosis (IPF), to screen candidate drugs for treating pulmonary fibrosis, in particular, idiopathic pulmonary fibrosis (IPF) in animals and human beings, and to search for drug targets of pulmonary fibrosis, in particular, idiopathic pulmonary fibrosis (IPF) of animals, and human beings.

In the first place, the present invention provides a method for constructing an animal model of pulmonary fibrosis, in particular, idiopathic pulmonary fibrosis (IPF), which comprises the step of increasing the mechanical tension on the alveolar epithelium of the animal.

Preferably, before the step of increasing the mechanical tension on the alveolar epithelium, the animal undergoes a pneumonectomy (PNX).

Preferably, the step of increasing the mechanical tension on the alveolar epithelium includes the step of increasing the mechanical tension on alveolar type II (AT2) cells.

Preferably, the step of increasing the mechanical tension on alveolar type II (AT2) cells involves the step of deactivating Cdc42 in AT2 cells (Cdc42 AT2 null). Deactivating Cdc42 in AT2 cells involves deleting, disrupting, inserting, knocking-out or inactivating Cdc42 genes in AT2 cells.

Preferably, the present invention provides a method for constructing an animal model of pulmonary fibrosis, in particular, idiopathic pulmonary fibrosis (IPF), which comprises the step of knocking-out Cdc42 gene in AT2 cells, preferably, in PNX-treated animal.

The loss of Cdc42 gene in AT2 cells leads to progressive lung fibrosis in PNX-treated animals. Moreover, this progressive lung fibrosis phenotype also occurs in non-PNX-treated Cdc42 AT2 null animals in middle age and old age.

In the lungs of Cdc42 AT2 null animals, fibroblastic foci are developed.

Preferably, the animal may be mouse, rabbit, rat, canine, pig, horse, cow, sheep, monkey or chimpanzee.

In the second place, the present invention provides an animal model constructed through increasing the mechanical tension on the alveolar epithelium of the animal.

Preferably, the present invention provides an animal model constructed through increasing the mechanical tension on AT2 cells of the animal.

Preferably, the present invention provides an animal model of pulmonary fibrosis, in particular, idiopathic pulmonary fibrosis (IPF), wherein the mechanical tension on the alveolar epithelium of the animal is increased.

Preferably, the present invention provides an animal model of pulmonary fibrosis, in particular, idiopathic pulmonary fibrosis (IPF), wherein Cdc42 gene in AT2 cells is deactivated.

Preferably, the present invention provides an animal model of pulmonary fibrosis, in particular, idiopathic pulmonary fibrosis (IPF), wherein Cdc42 gene in AT2 cells is deleted, disrupted, inserted, knocked-out or inactivated.

Preferably, the present invention provides an animal model of pulmonary fibrosis, in particular, idiopathic pulmonary fibrosis (IPF), wherein Cdc42 gene in AT2 cells is knocked out.

Preferably, the present invention provides an animal model of pulmonary fibrosis, in particular, idiopathic pulmonary fibrosis (IPF), wherein the animal model shows progressive lung fibrosis phenotype after undergoing PNX. Moreover, the present invention provides an animal model of pulmonary fibrosis, in particular, idiopathic pulmonary fibrosis (IPF), wherein the animal model without undergoing PNX shows progressive lung fibrosis phenotype in middle age and old age.

In the disease animal model of pulmonary fibrosis, in particular, idiopathic pulmonary fibrosis (IPF) in the present invention, fibroblastic foci are developed.

Preferably, the present animal model develops fibrotic changes after the pneumonectomy (PNX) treatment.

Preferably, the present animal model shows genotype of Cdc42 AT2 null.

Preferably, the present animal model is Cdc42 AT2 null mouse.

Preferably, the animal may be mouse, rabbit, rat, canine, pig, horse, cow, sheep, monkey or chimpanzee.

In the third place, the present invention provides an AT2 cell of lung, wherein the mechanical tension on the alveolar epithelium is increased.

Preferably, the present invention provides an AT2 cell, wherein Cdc42 gene is deactivated. Preferably, the present invention provides an AT2 cell, wherein Cdc42 gene is knocked out. Preferably, the present invention provides a Cdc42 null AT2 cell.

In the fourth place, the present invention provides a lung, wherein the mechanical tension on the alveolar epithelium is increased.

Preferably, the present invention provides a lung, wherein Cdc42 gene in the AT2 cells of the lung is deactivated. Preferably, Cdc42 gene in the AT2 cells of the lung is knocked out. Preferably, the present invention provides a lung having Cdc42 null AT2 cells.

Preferably, the said lung is obtained by using a Spc-CreER allele to knockout Cdc42 specifically in lung AT2 cells (pulmonary alveolar stem cells).

In the fifth place, the present invention provides a method for screening candidate drugs for treating pulmonary fibrosis, in particular, idiopathic pulmonary fibrosis (IPF) of animals and human beings using the said animal model.

In the sixth place, the present invention provides use of the said animal model or cultured AT2 cells thereof in searching for a drug target aiming at treating pulmonary fibrosis, in particular, idiopathic pulmonary fibrosis (IPF) of animals and human beings.

Preferably, the present invention searched out one kind of drug target, involving a positive feedback loop of TGFIβ/SMAD signaling in human or mouse AT2 cells. Preferably, the autocrine TGFβin human or mouse AT2 cells can activate TGFβ/SMAD signaling in these AT2 cells. Preferably, mechanical stretching can significantly increase the expression level of autocrine TGFβin both human and mouse AT2 cells. Preferably, the positive feedback loop of TGFβ/SMAD signaling in stretched human and mouse AT2 cells further results in the increased expression level of autocrine TGFβ.

In the seventh place, the present invention provides a method for evaluating the therapeutic effects of pulmonary fibrosis, in particular, idiopathic pulmonary fibrosis (IPF) using the said animal model.

In the eighth place, the present invention provides a method for the prognosis evaluation of pulmonary fibrosis, in particular, idiopathic pulmonary fibrosis (IPF) using the said animal model.

In the ninth place, the present invention provides use of the said animal model for screening candidate drugs for treating pulmonary fibrosis, in particular, idiopathic pulmonary fibrosis (IPF) of animals and human beings.

In the tenth place, the present invention provides a method for detecting the said animal model using a pair of primers designed on the basis of the sequences shown by SEQ ID NO:4.

Preferably, the primers for detecting the said animal model are shown as followed:

Forward:
(SEQ ID NO: 1)
CTGCCAACCATGACAACCTAA;
Reverse:
(SEQ ID NO: 2)
AGACAAAACAACAAGGTCCAG.

The Cdc42 null AT2 cells are unable to differentiate into ATI cells and thus cannot regenerate new alveoli after lung injuries, the alveolar epithelium of Cdc42 AT2 null mice continues to experience elevated mechanical tension.

The invention encompasses all combination of the particular embodiments recited herein.

Brief Description of the Drawings

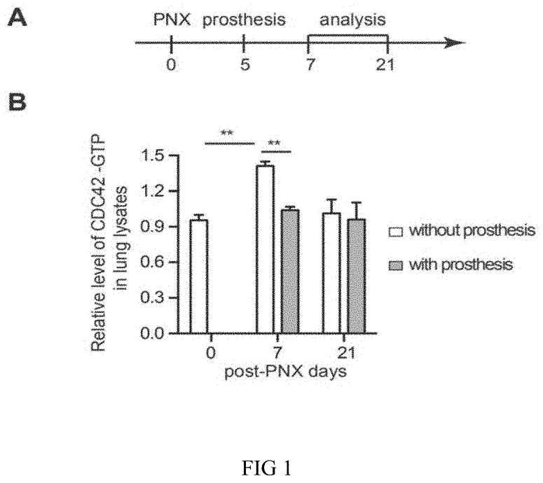

FIG. 1 shows the expression level of CDC42-GTP (the active form of CDC42) increases significantly at post-PNX day 7, which is the time when AT2 cells differentiate into AT1 cells.

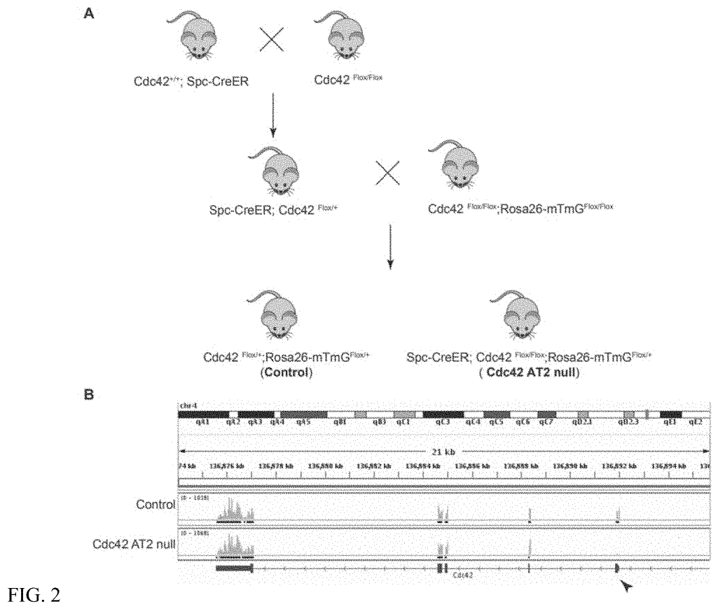

FIG. 2 shows the scheme of generating a mouse line in which Cdc42 gene is specifically deleted in AT2 cells.

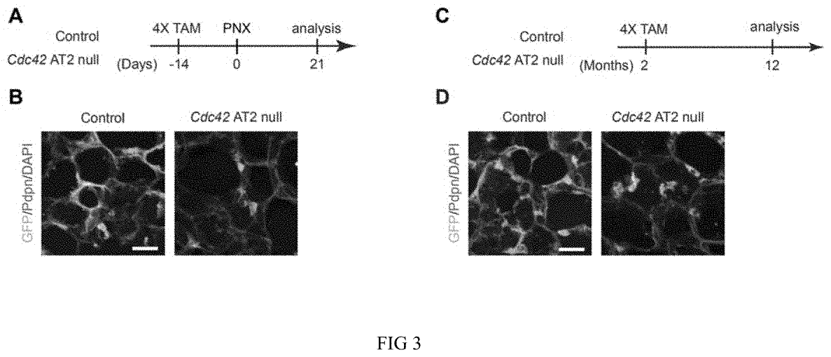

FIG. 3 shows that loss of Cdc42 gene in AT2 cells impairs the differentiation of AT2 cells during post-PNX alveolar regeneration or alveolar homeostasis

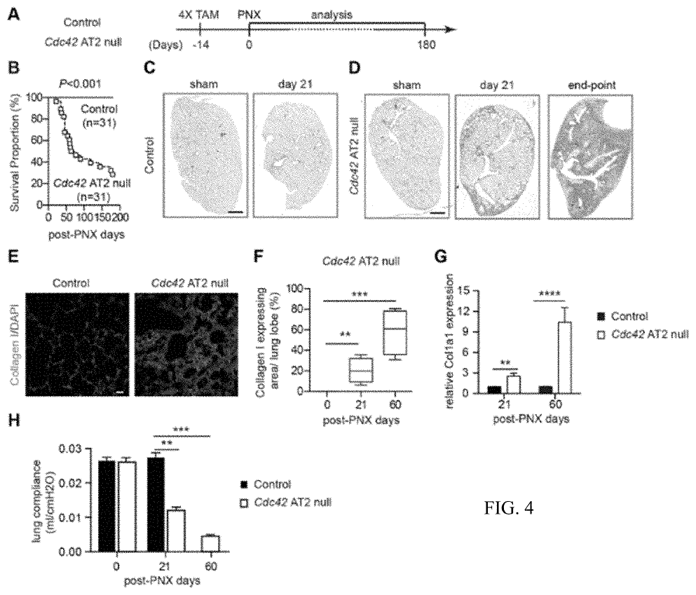

FIG. 4 shows that loss of Cdc42 in AT2 cells leads to progressive lung fibrosis in PNX-treated mice.

FIG. 5 shows that loss of Cdc42 in AT2 cells leads to progressive lung fibrosis in non- PNX-treated aged mice

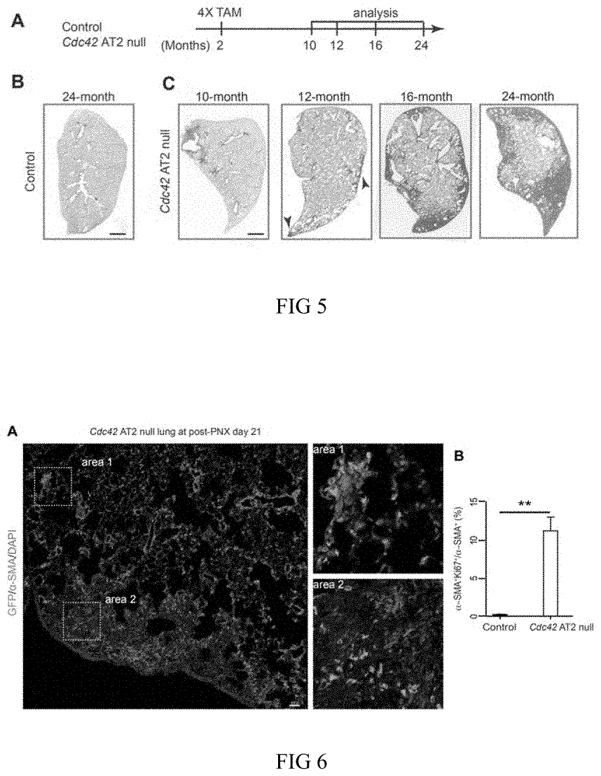

FIG. 6 shows the development of a-SMA⁺fibroblastic foci in the lungs of PNX- treated Cdc42 AT2 null mice.

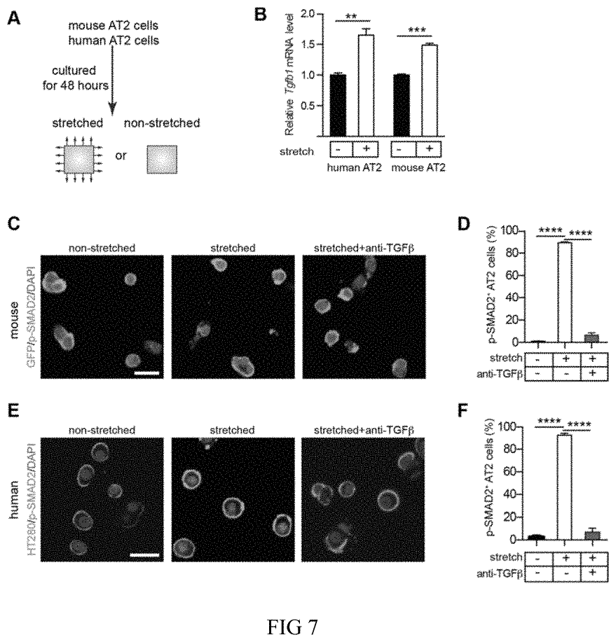

FIG. 7 shows that elevated mechanical tension activates an autocrine TGFβsignaling in mouse and human AT2 cells.

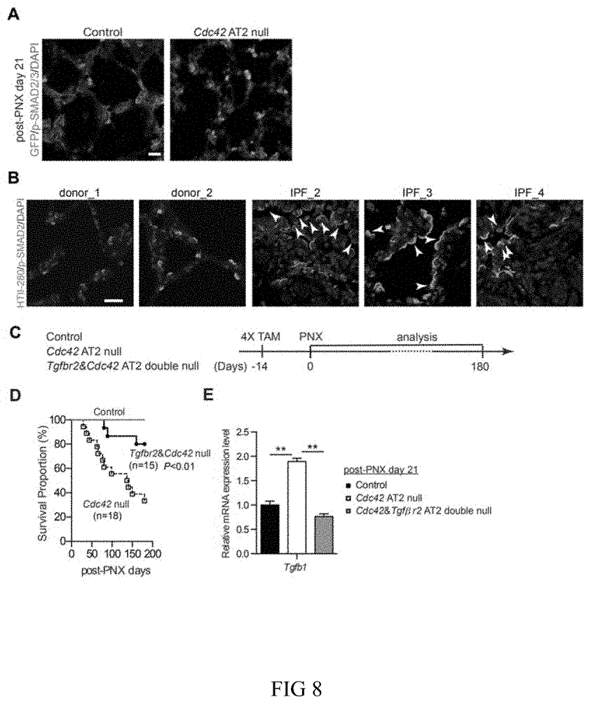

FIG. 8 shows increased TGFβsignaling in AT2 cells of PNX-treated Cdc42 AT2 null mice and IPF patients. And decreasing TGFβsignaling in AT2 cells of PNX-treated Cdc42 AT2 null mice attenuating the fibrosis development.

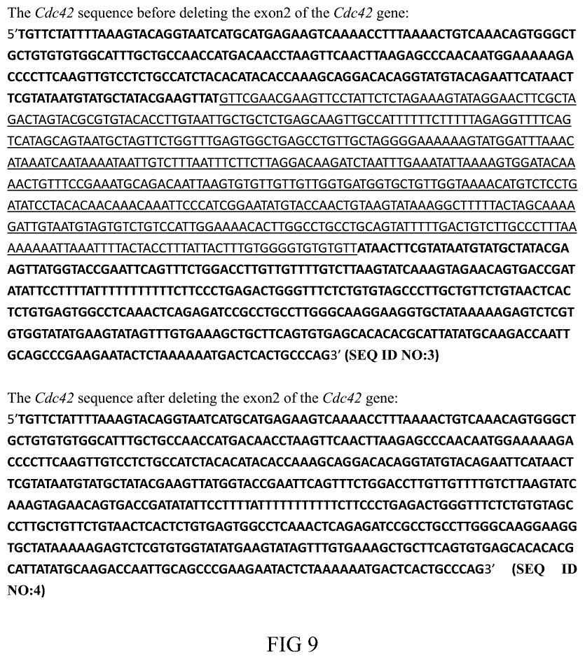

FIG. 9 shows the fragments of Cdc42 DNA sequence before and after deleting the exon2 of the Cdc42 gene.

Description of Particular Embodiments of the Invention

The descriptions of particular embodiments and examples are provided by way of illustration and not by way of limitation. Those skilled in the art will readily recognize a variety of noncritical parameters that could be changed or modified to yield essentially similar results.

The idiopathic pulmonary fibrosis (IPF) is a type of chronic lung disease characterized by a progressive and irreversible decline in lung function. Symptoms typically include gradual onset of shortness of breath and a dry cough. Other changes may include feeling tired and nail clubbing. Complications may include pulmonary hypertension, heart failure, pneumonia, or pulmonary embolism.

The alveolar epithelia of lungs are composed of a combination of both alveolar type I (AT1) and type II (AT2) cells. AT2 cells are the alveolar stem cells, and can differentiate into AT1 cells during alveolar homeostasis and post-injury repair. AT1 cells-which ultimately comprise fully 95% of the alveolar surface in adult lungs-are large squamous cells that function as the epithelial component of the thin air-blood barrier¹⁴. In IPF tissues, abnormal hyperplastic AT2 cells are typically located adjacent to fibroblastic foci'^{s}, and the gene mutants that affect the functions of AT2 cells are frequently observed in IPF tissues in the clinic¹⁶′¹⁷. The precise pathological mechanisms underlying abnormal AT2 physiology and progressive pulmonary fibrosis remain to be elucidated.

“Animal model” or “disease animal model” is a living, non-human animal used for research and investigation of human diseases, for the purpose of better understanding the disease process, the pathological mechanisms, and for the purpose of screening effective drugs and searching for ideal drug targets.

Searching for potential drug target(s) for a disease is the first step in the discovery of a drug, and is also the key point for screening new drugs for a disease.

In an embodiment of the present invention, based on the findings that the expression level of CDC42-GTP in the post-PNX lungs (having significantly increases mechanical tension) is increased significantly (FIGS. 1A and 1B), and such increased expression of CDC42-GTP can be inhibited by a prosthesis implantation (FIGS. 1A and 1B), the effect of deleting Cdc42 genes in AT2 cells during PNX-induced alveolar regeneration is investigated in the present invention. **P<0.01, Student's t test.

The Sftpc gene promoter-driven recombinase (Spc-CreER) is used to specifically delete genes in AT2 cells after administration of tamoxifen to the animal. The CreER mouse system is commonly used for inducible gene knockout studies.

In an embodiment of the present invention, a mouse line in which Cdc42 gene is specifically deleted in AT2 cells is constructed. The mouse in the present invention is named as Cdc42 AT2 null mice (FIG. 2). In the lungs of Cdc42 AT2 null mice, few AT2 cells differentiated into AT1 cells, and no new alveoli are formed at post-PNX day 21 (FIG. 3B).

In an embodiment of the present invention, some Cdc42 AT2 null mice showed significant weight loss and increased respiration rates after PNX treatment day 21 (FIGS. 4A and 4B). Indeed, fully 50% of PNX-treated Cdc42 AT2 null mice reached the predefined health- status criteria for endpoint euthanization by post-PNX day 60 (FIG. 4B), and about 80% of PNX-treated Cdc42 AT2 null mice reached their endpoints by post-PNX day 180 (FIG. 4B). Cdc42 AT2 null mice revealed severe fibrosis in their lungs at their endpoints (FIG. 4D).

In an embodiment of the present invention, Cdc42 AT2 null mice after PNX reveal severe fibrosis in the lungs of Cdc42 AT2 null mice at their endpoints (FIG. 4D compared with FIG. 4C). The subpleural regions of some Cdc42 AT2 null lungs exhibit signs of tissue thickening starting from post-PNX day 21 (FIG. 4D). By the end-point, the dense fibrosis has progressed to the center of most Cdc42 AT2 null lungs.

In an embodiment of the present invention, Collagen I is detected in the dense fibrotic regions in the lungs of Cdc42 AT2 null mice (FIG. 4E), and the proportion of Collagen I expressing area per lobe gradually increases in Cdc42 AT2 null mice after PNX treatment (FIG. 4F). The qPCR analysis also shows that the Collagen I mRNA expression level increases gradually from post-PNX day 21 (FIG. 4G). Moreover, gradually decreased lung compliance is observed in PNX-treated Cdc42 AT2 null mice from post-PNX day 21 as compared to their PNX-treated Control mice (FIG. 4H). Decreased lung compliance is known to occur frequently as lungs become fibrotic¹⁹⁻²⁴.

In an embodiment of the present invention, Cdc42 AT2 null mice without PNX treatment from 10-months of age to 24-months of age (FIG. 5A) show that no significant fibrotic changes before the Cdc42 AT2 null mice reach 10-months of age (FIG. 5C). Fibrotic changes in the lungs of control mice are never observed, even the control mice reached 24- months of age (FIG. 5B), but, by 12 months, fibrosis have obviously begun to develop in the subpleural regions of Cdc42 AT2 null lungs and to progress toward the center of the lung (FIG. 5C).

Fibroblastic foci are considered as a relevant morphologic marker of progressive pulmonary fibrosis and are recognized as sites where fibrotic responses are initiated and/or perpetuated in progressive pulmonary fibrosis. The fibroblastic foci contain proliferating α- SMA⁺fibroblasts.

In an embodiment of the present invention, it is observed that some α-SMA⁺fibroblasts start to accumulate next to a cluster of AT2 cells in the relative normal alveolar regions of Cdc42 AT2 null lungs (area 1, FIG. 6A). And the dense fibrosis region of the lungs is filled with α-SMA⁺fibroblasts (area 2, FIG. 6A). In addition, the cell proliferation of α-SMA⁺cells increases dramatically in the lungs of Cdc42 AT2 null mice at post-PNX day 21, indicating that the proliferating α-SMA⁺fibroblasts contribute to the development of lung fibrosis (FIG. 6B).

### EXAMPLES

Methods

Mice and survival curve record.

Rosa26-CAG-mTmG (Rosa26-mTmG), Cdc42 ^{flox/flox} mice²⁵, and Tgfbr2 mice²⁶ have been described previously. All experiments were performed in accordance with the recommendations in the Guide for Care and Use of Laboratory Animals of the National Institute of Biological Sciences. To monitor the survival of mice, both the Control and the Cdc42 AT2 null mice were weighed every week after the PNX treatment. Once the mice reached the pre- defined criteria for end-points, the mice were sacrificed. We define the endpoints according to the pre-defined criteria^{27,28}.

Generating Spc-CreER;rtTA (Spc-CreER) knock-in mice.

The CreERT2, p2a, and rtTA element were enzyme-linked and inserted into the mouse endogenous SPC gene. The insertion site is the stop codon of the endogenous SPC gene, then a new stop codon was created at the 3′ end of rtTA. The CRISPR/Cas9 technology was used to insert the CreERT2-p2a-rtTA fragment into the genome.

Pneumonectomy (PNX) and prosthesis implantation.

The male mice of 8 weeks old were injected with tamoxifen (dosage: 75mg/kg) every other day for 4 times. The mice were anesthetized and connected to a ventilator (Kent Scientific, Topo) from 14^{th} day after the final dose of tamoxifen injection. The chest wall was incised at the fourth intercostal ribs and the left lung lobe was removed. For prosthesis implantation, a soft silicone prosthesis with a similar size and shape of the left lung lobe was inserted into the empty left lung cavity.

Pulmonary function test.

Lung function parameters were measured using the invasive pulmonary function testing system (DSI Buxco® PFT Controller). Mice were first anesthetized before inserting an endotracheal cannula into their trachea. The dynamic compliance results were obtained from Resistance & Compliance Test. The forced vital capacity results were obtained from the Pressure Volume Test.

Hematoxylin and Eosin (H&E) staining and immunostaining.

Lungs were inflated with 4% paraformaldehyde (PFA) and were continually fixed in 4% PFA at 4° C. for 24 hours. Then the lungs were cryoprotected in 30% sucrose and embedded in OCT (Tissue Tek).

The H&E staining experiment followed the standard H&E protocol. Briefly, slides were washed by water to remove the OCT. The nuclei were stained by hemotoxylin (Abcam, ab150678) for 2 minutes and the cytoplasm was stained by eosin (Sigma, HT110280) for 3 minutes. Slices were sealed with neutral resin after the dehydration and clearing steps.

The immunofluorescence staining experiments followed the protocol previously described²⁹. In brief, after removing the OCT, the lung slices were blocked with 3%BSA/0.1%TritonX-100/PBS for 1 hour, then slides were incubated with primary antibodies at 4° C. for overnight. After washing the slides with 0.1%TritonX-100/PBS for 3 times, the slices were incubated with secondary antibodies for 2 hours at room temperature.

The primary antibodies used in the paper are listed below:

| Name | Company and catalog number | Dilution |
|---|---|---|
| Chicken anti-GFP | Abcam, ab13970-100 | 1:500 |
| Rabbit anti-Collagen I | Abcam, ab34710 | 1:300 |
| Mouse anti a-SMA | Sigma, C6198 | 1:300 |
| Rabbit anti pSmad2 | CST, #3101 | 1:500 |
| Mouse anti HT2-280 | Terrace Biotech, TB-27AHT2-280 | 1:50 |
| Hamster anti-Pdpn | Developmental Studies Hybridoma | 1:100 |
| | Bank, clone8.1.1 | |

The secondary antibodies used in the paper are listed below:

| Name | Company and catalog number | Dilution |
|---|---|---|
| Alexa Fluor 488 Donkey | 703-545-155, Jackson Immuno | 1:500 |
| anti-Chicken | Research | |
| Alexa Fluor 488 Donkey | 715-545-150, Jackson Immuno | 1:500 |
| anti-mouse | Research | |
| Alexa Fluor 568 Donkey | A11057, Invitrogen | 1:500 |
| anti-rabbit | | |
| Alexa Fluor 647 Goat | A-21451, Invitrogen | 1:500 |
| anti-hamster | | |
| Biotin Donkey Anti- | 711-065-152, Jackson Immuno | |
| Rabbit | Research | |

For the p-SMAD2 staining experiment, 1X phosphatase inhibitor (Bimake, B15002) was added in 4% PFA during the tissue fixation process. The tyramide signal amplification method was used for pSMAD2 staining.

The human lung tissues were fixed with 4% PFA for 24 hours at 4° C., cryoprotected in 30% sucrose and embedded in OCT. All experiments were performed with the Institutional Review Board approval at both National Institute of Biological Sciences and China-Japan Friendship Hospital.

Statistical analysis.

All data are presented as mean ±s.e.m. (as indicated in figure legends). The data presented in the figures were collected from multiple independent experiments that were performed on different days using different mice. Unless otherwise mentioned, most of the data presented in figure panels are based on at least three independent experiments. The inferential statistical significance of differences between sample means was evaluated using two-tailed unpaired Student's t-tests.

Isolating mouse AT2 cells.

After 4 doses of tamoxifen injection, the lungs of Spc-CreER, Rosa26-mTmG mice were dissociated as previously described^{19,44}. Briefly, anesthetized mice were inflated with neutral protease (Worthington-Biochem, LS02111) and DNase I (Roche, 10104159001). AT2 cells were directly sorted based on the GFP fluorescence using the single-cell-select-mode in BD FACS Aria II and III appliances.

Isolating human AT2 cells.

The human lung tissues were cut into small pieces with a scalpel, then digested by neutral protease (Worthington-Biochem, LS02111), DNase I (Roche, 10104159001), collagenase type I (Gibco, 17100-017) and elastase (Worthington, 2294). Then the digested suspension was sorted for CD326⁺, HTII-280⁺CD45⁻, CD31⁻ cells using the single-cell-select- mode in BD FACS Aria II and III appliances. All experiments were performed with the Institutional Review Board approval at both National Institute of Biological Sciences, Beijing and China-Japan Friendship Hospital, Beijing.

Primary human and mouse AT2 cell culture and cell stretching assay.

Primary AT2 cells were sorted by FACS and plated on silicone membranes for 24 hours before performing the stretching experiments. The equiaxial strain system and methods were previously described in details³⁰. 24 hours after performing a static stretch with a 25% change in surface area, primary AT2 cells were assayed for anti-p-SMAD2 staining. To culture with a TGFβneutralizing antibody (biolegend, 521703) with stretched human or mouse AT2 cells, 1μg/ml TGFβneutralizing antibody was added in the culture medium.

Quantitative RT-PCR (qPCR).

Total RNA was isolated from either whole lung or primary AT2 cells using Zymo Research RNA Mini Prep Kits (R2050). Reverse transcription reactions were performed with a two-step cDNA synthesis Kit (Takara, Cat. # 6210A/B) according to the manufacturer's recommendations. qPCR was done with a CFX96 Touch™Real-Time PCR Detection System. The mRNA levels of target genes were normalized to the Gapdh mRNA level.

Primers used for qPCR are listed below.

| | Forward | Reverse |
|---|---|---|
| Gapdh | AAGGTCGGTGTGAACGGAT | CGTTGAATTTGCCGTGAGT |
| | TTGG(SEQ ID NO: 5) | GGAG(SEQ ID NO: 6) |
| Col1a1 | CCTCAGGGTATTGCTGGAC | CAGAAGGACCTTGTTTGCC |
| | AAC(SEQ ID NO: 7) | AGG(SEQ ID NO: 8) |
| Human | TACCTGAACCCGTGTTGCT | GTTGCTGAGGTATCGCCAG |
| Tgfb1 | CTC(SEQ ID NO: 9) | GAA(SEQ ID NO: 10) |
| Mouse | TGATACGCCTGAGTGGCTG | CACAAGAGCAGTGAGCGCT |
| Tgfb1 | TCT(SEQ ID NO: 11) | GAA(SEQ ID NO: 12) |

3D alveolar reconstruction.

For vibratome sections, lungs were gently inflated to full capacity with 2% low-melting agarose. Then lungs were fixed in 4% PFA for overnight at 4° C. Thick vibratome sections were sliced at a thicknesses of 200μm using the vibrating microtome (Leica VTIOOS). Immunostaining experiments were performed as the standard wholemount staining protocol. Z stack images were taken by Leica LSI macro confocal microscope and/or A1-R inverted confocal microscope.

CDC42-GTP assay.

The GTP-CDC42 level is determined using the CDC42 activation assay biochem kit (cytoskeleton, #BK127) according to the provided manufacturer's recommendations. Briefly, the whole lung lobes were grinded in liquid nitrogen, then lysed using the cell lysis buffer (applied in the kit). Then the cell lysates were added into the microplate wells applied. After the reaction, the absorbance at 490nm was measured.

Primer sequences for sequencing the fragment of Cdc42 DNA sequence before and after deleting the exon2 of the Cdc42: Forward: CTGCCAACCATGACAACCTAA(SEQ ID NO:1) ; Reverse: AGACAAAACAACAAGGTCCAG(SEQ ID NO:2).

Example 1. Generating a mouse line in which Cdc42 gene is specifically deleted in AT2 cells

1. In order to construct a progressive lung fibrosis animal model, Cdc42 AT2 null mice are generated by knocking out Cdc42 gene specifically in alveolar type II cells (AT2 cells).

In order to specifically delete Cdc42 gene in AT2 cells, mice carrying a Spc-CreER knock-in allele are crossed with Cdc42 floxed (Cdc42^{flox/flox}) mice (FIG. 2A). In Cdc42 ^{flox/flox} mice, the exon 2 of Cdc42 gene, which contains the translation initiation exon of Cdc42 gene, is flanked by two loxp sites. In Spc-CreER; Cdc42 ^{flox/flox} mice the exon 2 of Cdc42 gene, exon 2 of Cdc42 gene is specifically deleted in AT2 cells by Cre/loxp-mediated recombination after tamoxifen treatment (FIG. 2B). Spc-CreER; Cdc42^{flox/flox} mice are named as Cdc42 AT2 null mice.

2. Lungs of Cdc42 AT2 null mice develop progressive fibrotic changes after PNX treatment.

Left lung lobe resection (peumonectomy, PNX) on Cdc42 AT2 null mice and control mice were performed. The lungs of Cdc42 AT2 null mice and control mice at different time points after PNX treatment were analyzed (FIG. 4A). We found that some Cdc42 AT2 null mice showed significant weight loss and increased respiration rates after post-PNX day 21. Indeed, fully 50% of PNX-treated Cdc42 AT2 null mice reached the predefined health-status criteria for endpoint euthanization by post-PNX day 60 (FIG. 4B), and more than 70% of PNX-treated Cdc42 AT2 null mice (n=33) reached their endpoints by post-PNX day 180 (FIG. 4B). H&E staining shows lungs of sham-treated and PNX-treated control mice do not shown fibrotic changes (FIG. 4C). H&E staining shows that lungs of PNX-treated Cdc42 AT2 null mice at endpoints have significantly increased fibrotic area than the lungs at post-PNX day 21 (FIG. 4D).

3. Developing fibrotic changes at the edge of lungs of Cdc42 AT2 null mice at post- PNX day 21.

The lungs of Cdc42 AT2 null mice start to show fibrotic changes at post-PNX day 21. The Spc-Cdc42^{flox/-}lungs have shown dense fibrotic changes at the edge of lungs (FIG. 4D). H&E staining shows that histological changes of the fibrotic region of Cdc42 AT2 null lungs recapitulates the histological changes of human IPF lungs.

4. Characterizing the collagen I deposition in fibrotic lungs, and analyzing lung compliance.

Lungs collected from Control and Cdc42 AT2 null mice at post-PNX day 21 were stained with an anti-Collagen I antibody (FIG. 4E). Much stronger immunofluorescence signals for Collagen I are detected in the dense fibrotic regions of lungs of Cdc42 AT2 null mice as compared with control lungs. The area of dense Collagen I in lungs of Cdc42 AT2 null mice gradually increases from post-PNX day 21 to post-PNX day 60 (FIG. 4F). qPCR analysis showed that the Collagen I mRNA expression levels increased gradually from post-PNX day 21 to post-PNX day 60 in lungs of Cdc42 AT2 null mice (FIG. 4G). *P<0.05, ***P<0.001; ****P<0.0001, Student's t test.

The lung compliance of lungs of Cdc42 AT2 null mice gradually decreases after PNX.

5. Developing progressive lung fibrosis in no-PNX-treated Cdc42 AT2 null mice starting from around 12 months of age.

Control and Cdc42 AT2 null mice were exposed to 4 doses of tamoxifen 14 days starting at age of 2 months. Lungs of Control and Cdc42 AT2 null mice without PNX treatment were collected at 10, 12, 16, or 24 months (FIG. 5A). The lungs of Control and Cdc42 AT2 null mice without PNX treatment were analyzed and found no significant fibrotic changes before the Cdc42 AT2 null mice reached 10-months of age (FIGS. 5B and 5C). By 12 months, fibrosis had obviously begun to develop in the subpleural regions of Cdc42 AT2 null lungs and to progress toward the center of the lung (FIG. 5C). Thus, the loss of Cdc42 in AT2 cells leads to progressive lung fibrosis in no-PNX-treated Cdc42 AT2 null mice starting from around 12 months of age.

6. Characterization of the development of α-SMA⁺fibroblastic foci in the lungs of Cdc42 AT2 null mice.

Fibroblastic foci are considered a relevant morphologic marker of progressive pulmonary fibrosis and are recognized as sites where fibrotic responses are initiated and/or perpetuated in progressive pulmonary fibrosis³¹ . The fibroblastic foci contain proliferating α- SMA⁺fibroblasts³² . Lungs of Cdc42 AT2 null mice at post-PNX day 21 were stained with antibodies against α-SMA (FIG. 6A). Some α-SMA⁺fibroblasts started to accumulate next to a cluster of AT2 cells in the relative normal alveolar regions of Cdc42 AT2 null lungs (area 1, FIG. 6A). And the dense fibrosis region of the lungs is filled with α-SMA⁺fibroblasts (area 2, FIG. 6A). In addition, the cell proliferation of α-SMA⁺cells increased dramatically in the lungs of Cdc42 AT2 null mice at post-PNX day 21 by immunostaining using antibodies against both α-SMA and proliferation marker, Ki67. These results indicate that the proliferating α- SMA⁺fibroblasts contribute to the development of lung fibrosis (FIG. 6B). **P<0.01, Student's t test.

Example 2. Sequence characterization of the Cdc42 AT2 null mice

The Spc-CreER, Cdc42 ^{flox/-} mice were performed genome purification and PCR amplification. Then the fox and null bands of Cdc42 were purified and sequenced using the primers as below: CTGCC AA CC ATG AC AACCTAA (SEQ ID NO: 1); AGACAAAACAACAAGGTCCAG (SEQ ID NO:2).

The fragments of Cdc42 DNA sequence before or after deleting the exon2 of the Cdc42 gene are shown in FIG. 9.

Examples 1 and 2 demonstrate that Cdc42 AT2 null mice are exactly the disease animals of progressive pulmonary fibrosis, in particular, IPF. The following examples show the features of the Cdc42 AT2 null mice, and the uses of the Cdc42 AT2 null mice.

Example 3. Cdc42 is essential for the differentiation of AT2 cells during post-PNX alveolar regeneration or under normal alveolar homeostasis conditions.

We performed PNX on control and Cdc42 AT2 null mice and analyzed the alveolar regeneration and AT2 cell differentiation at post-PNX day 21. As shown in FIG. 3A, 200μm lung sections of control and Cdc42 AT2 null mice are immunostained with antibodies against GFP, Pdpn, and Prospc. At post-PNX day 21, many newly differentiated AT1 cells and newly formed alveoli are observed in no-prosthesis-implanted control lungs (FIG. 3B). However, in Cdc42 AT2 null lungs, few AT2 cells have differentiated into AT1 cells, and no new alveoli are formed at post-PNX day 21 (FIG. 3B). It is observed that the alveoli in peripheral region of the Cdc42 AT2 null lungs are profoundly overstretched (FIG. 3B).

Under normal homeostatic conditions, AT2 cells slowly self-renew and differentiate into AT1 cells to establish new alveoli. To examine whether Cdc42 is required for AT2 cell differentiation during homeostasis, we deleted Cdc42 in AT2 cells when the mice were two- months old and analyzed the fate of AT2 cells until the mice were 12-month old. Lungs of Control and Cdc42 null mice without PNX treatment were collected at 12 months (FIG. 3C). Images show the maximum intensity of a 200μm Z-projection of lung sections that were stained with antibodies against GFP, Pdpn, and Prospc. In the lungs of 12-month Control mice, we observed formation of many new alveoli (FIG. 3D). However, in the lungs of 12-month Cdc42 null mice (that had not undergone PNX), we observed enlarged alveoli with lacking any new AT1 cell formation (FIG. 3D).

Example 4. Loss of Cdc42 in AT2 cells leads to progressive lung fibrosis in PNX- treated mice

Cdc42 AT2 null and control mice after PNX are observed for a longer period of time (FIG. 4A). Surprisingly, some Cdc42 AT2 null mice showed significant weight loss and increased respiration rates after post-PNX day 21. Indeed, fully 50% of PNX-treated Cdc42 AT2 null mice reached the predefined health-status criteria for endpoint euthanization by post-PNX day 60 (FIG. 4B), and about 80% of PNX-treated Cdc42 AT2 null mice reached their endpoints by post-PNX day 180 (FIG. 4B).

H&E staining of post-PNX control and Cdc42 AT2 null mice reveals severe fibrosis in the lungs of Cdc42 AT2 null mice at their endpoints (FIG. 4D compared with FIG. 4C). In order to determine the point at which Cdc42 AT2 null mice begin to develop lung fibrosis following PNX, the lungs of Cdc42 AT2 null mice are analyzed at various time points after PNX using H&E staining (FIG. 4D). The subpleural regions of some Cdc42 AT2 null lungs exhibit signs of tissue thickening by post-PNX day 21 (FIG. 4D). By the end-point, the dense fibrosis has progressed to the center of most Cdc42 AT2 null lungs (FIG. 4D). What we have observed in post-PNX and aged Cdc42 AT2 null mice is similar to the characteristic progression of IPF, in which fibrotic lesions first occur at the lung periphery and subsequently progress inward towards the center of lung lobes.

In addition to detecting strong immunofluorescence signals for Collagen I in these dense fibrotic regions of lungs of Cdc42 AT2 null mice (FIG. 4E), we observe the proportion of Collagen I expressing area per lobe gradually increased after PNX in Cdc42 AT2 null mice (FIG. 4F). Our qPCR analysis also shows that the Collagen I mRNA expression levels increase gradually from post-PNX day 21 (FIG. 4G). Moreover, gradually decreased lung compliance is observed in PNX-treated Cdc42 AT2 null mice from post-PNX day 21 as compared to their PNX-treated Control mice (FIG. 4H), an intriguing finding given that decreased lung compliance is known to occur frequently as lungs become fibrotic¹⁹⁻²⁴.

Example 5. Loss of Cdc42 in AT2 cells leads to progressive lung fibrosis in non- PNX-treated aged mice

Since it is found that impaired AT2 differentiation and enlarged alveoli in 12-month old Cdc42 AT2 null mice (FIG. 3D), then lungs of control and Cdc42 AT2 null mice without PNX treatment are analyzed from 10-months of age to 24-months of age (FIG. 5A). Fibrotic changes in the lungs of control mice are never observed, even the control mice reached 24- months of age (FIG. 5B). We found no significant fibrotic changes before the Cdc42 AT2 null mice reached 10-months of age (FIG. 5C). It is also observed that by 12 months, fibrosis has obviously begun to develop in the subpleural regions of Cdc42 AT2 null lungs and to progress toward the center of the lung after 12 months (FIG. 5C).

Collectively, the loss of Cdc42 in AT2 cells leads to progressive lung fibrosis in PNX- treated mice. Moreover, this progressive lung fibrosis phenotype also occurs in no-PNX-treated Cdc42 AT2 null mice starting from around 12 months of age. All these results demonstrate that deletion of Cdc42 in AT2 cells leads to IPF like progressive pulmonary fibrosis in mice, and therefore, a mouse model of IPF like progressive lung fibrosis is established and can be used to study human IPF disease.

Example 6. The development of α-SMA⁺fibroblastic foci in the lungs of Cdc42 AT2 null mice

Fibroblastic foci are considered a relevant morphologic marker of progressive pulmonary fibrosis and are recognized as sites where fibrotic responses are initiated and/or perpetuated in progressive pulmonary fibrosis. The fibroblastic foci contain proliferating α- SMA⁺fibroblasts. Lungs of Cdc42 AT2 null mice at post-PNX day 21 are stained with antibodies against α-SMA (FIG. 6A). Some α-SMA⁺fibroblasts started to accumulate next to a cluster of AT2 cells in the relative normal alveolar regions of Cdc42 AT2 null lungs are observed (area 1, FIG. 6A). And the dense fibrosis region of the lungs is filled with α-SMA⁺fibroblasts (area 2, FIG. 6A). In addition, by immunostaining using antibodies against both α- SMA and proliferation marker, Ki67, we show that the cell proliferation of α-SMA⁺cells is increased dramatically in the lungs of Cdc42 AT2 null mice at post-PNX day 21. These results indicate that the proliferating α-SMA⁺fibroblasts contribute to the development of lung fibrosis in the lungs of Cdc42 AT2 null mice (FIG. 6B).

Example 7. Elevated mechanical tension caused by impaired alveolar regeneration leads to progressive lung fibrosis

The fact that lung fibrosis in Cdc42 AT2 null mice is greatly accelerated by the PNX treatment (FIG. 4) suggests a close link between lung fibrosis and mechanical tension-induced alveolar regeneration.

The loss of alveoli resulting from PNX substantially increases mechanical tension exerted upon the alveolar epithelium. The subsequent efficient regeneration of alveoli that occurs in normal mice eventually reduces the intensity of the mechanical tension to pre-PNX levels; however, as Cdc42 null AT2 cells are unable to differentiate into AT1 cells and thus cannot regenerate new alveoli (FIGS. 3A and 3B), the alveolar epithelium of Cdc42 AT2 null mice continue to experience elevated mechanical tension, which results in the progressive development of fibrosis (FIG. 4).

Example 8. Elevated mechanical tension activates a positive feedback loop of TGFβ/SMAD signaling in AT2 cells

Our results provide compelling evidence that elevated mechanical tension on the alveolar epithelium is critical for the progression of lung fibrosis. Using our previously established equibiaxial strain cell culture system, we cultured human or mouse AT2 cells on silicon membranes under either stretched or non-stretched condition (FIG. 7A). We have demonstrated that the application of mechanical tension to primary mouse and human AT2 cells can significantly increase the expression level of autocrine TGFβ, a fibrotic factor (FIG. 7B). To analyze whether the TGFβproduced by AT2 cells can activate the TGFβ/SMAD signaling in AT2 cells, we cultured human or mouse AT2 cells on silicon membranes under either stretched or non-stretched condition (FIG. 7A). We found that mechanical stretching can activate the TGFβ/SMAD signaling in both human and mouse AT2 cells (FIGS. 7C-7F). When we cultured stretched human or mouse AT2 cells with a TGFβneutralizing antibody, we found that the increased TGFβ/SMAD signaling in stretched human or mouse AT2 cells can be fully inhibited (FIGS. 7C-7F). These results indicate the autocrine TGFβin human or mouse AT2 cells can activate TGFβ/SMAD signaling in these AT2 cells. Together, these results demonstrate that a positive feedback loop of TGFβ/SMAD signaling in stretched AT2 cells further results in increased expression level of autocrine TGFβ. **P<0.01, Student's t test.

Therefore, the positive feedback loop of TGFβ/SMAD signaling in AT2 cells will be an ideal drug target for screening candidate drugs for pulmonary fibrosis, in particular, idiopathic pulmonary fibrosis (IPF).

Example 9. Reducing TGFβsignaling in AT2 cells attenuate progression of lung fibrosis

To further assess the activity of TGFβsignaling in lungs of Control and Cdc42 AT2 null mice at post-PNX day 21, we performed immunostaining experiments with an antibody against p-SMAD2 (FIG. 8A), an indicator of canonical TGFβsignaling activity. Whereas few of the AT2 cells in the Control lungs expressed nuclear p-SMAD2, many AT2 cells in Cdc42 null lungs expressed nuclear p-SMAD2 (FIG. 8A), indicating robust activation of TGFβsignaling in the AT2 cells of Cdc42 AT2 null mice at post-PNX day 21. Many AT2 cells in IPF specimens also expressed nuclear p-SMAD2, demonstrating the activation of TGFβsignaling in AT2 cells of IPF lungs (FIG. 8B).

It is well-established that the binding of TGFβligand to the TGFBR2 is essential for the activation of TGFβ/SMAD signaling³³. We generated Tgfbr2&Cdc42 AT2 double null mice, in which both Tgfbr2 and Cdc42 genes are deleted in AT2 cells. We performed left lung resection on Cdc42 AT2 null and Tgfbr2&Cdc42 AT2 double null mice and observed these mice for 180 days after PNX (FIG. 8C). Strikingly, 80% of the Tgfbr2&Cdc42 AT2 double null mice were alive by post-PNX day 180, while fewer than 40% of the Cdc42 AT2 null mice were alive by this time (FIG. 8D). These results provide compelling evidence that activated TGFβsignaling in AT2 cells drives the development of lung fibrosis in Cdc42 AT2 null mice. TGFβligand, Tgfb1, is one of downstream targets of TGFβ/SMAD signaling. By qPCR analysis, we found that the expression level of Tgfb1 is significantly increased in Cdc42 null AT2 cells but not in Tgfbr2&Cdc42 double null AT2 cells (FIG. 8E). This indicates that Cdc42 null AT2 cells produce more TGFβligands, due to the increased TGFβ/SMAD signaling. **P<0.01, Student's t test.

This example exactly shows that Cdc42 AT2 null mice may be used to find new drug target for IPF like progressive pulmonary fibrosis, and the TGFβsignaling in AT2 cells is such an ideal target.

Results:

To investigate the long-term effect(s) of impaired alveolar regeneration, we here observed Cdc42 AT2 null and littermate control (Control) mice for a longer period of time after left lung lobe resection (FIG. 4A). Surprisingly, we found that some Cdc42 AT2 null mice showed significant weight loss and increased respiration rates after post-PNX day 21. Indeed, fully 50% of PNX-treated Cdc42 AT2 null mice reached the predefined health-status criteria for endpoint euthanization by post-PNX day 60 (FIG. 4B), and more than 70% of PNX-treated Cdc42 AT2 null mice reached their endpoints by post-PNX day 180 (FIG. 4B).

H&E staining of post-PNX Control and Cdc42 AT2 null mice revealed severe fibrosis in the lungs of Cdc42 AT2 null mice at their endpoints (FIG. 4D compared with FIG. 4C). To determine the point at which Cdc42 AT2 null mice began to develop lung fibrosis following PNX, we analyzed the lungs of Cdc42 AT2 null mice at various time points after PNX using H&E staining (FIG. 4D). The subpleural regions of some Cdc42 AT2 null lungs exhibited signs of tissue thickening by post-PNX day 21 (FIG. 4D). By end-point, the dense fibrosis had progressed to the center of most Cdc42 AT2 null lungs.

In addition to detecting strong immunohistological signals for Collagen I in these dense fibrotic regions of lungs of Cdc42 AT2 null mice at post-PNX day 21 (FIG. 4E), the area of dense Collagen I in lungs of Cdc42 AT2 null mice gradually increases from post-PNX day 21 to post-PNX day 60 (FIG. 4F). qPCR analysis showed that the Collagen I mRNA expression levels increased gradually from post-PNX day 21 to post-PNX day 60 in lungs of Cdc42 AT2 null mice (FIG. 4G). *P<0.05, ***P<0.001; ****P<0.0001, Student's t test.

Additionally, there were significant reductions in lung compliance in the PNX-treated Cdc42 AT2 null mice as compared to their PNX-treated Control mice (FIG. 4H), an intriguing finding given that decreased FVC and decreased lung compliance are known to occur frequently as lungs become fibrotic¹⁹⁻²⁴.

We also analyzed the lungs of Control and Cdc42 AT2 null mice without PNX treatment and found no significant fibrotic changes before the Cdc42 AT2 null mice reached 10- months of age (FIGS. 5A-5C). By 12 months, fibrosis had obviously begun to develop in the subpleural regions of Cdc42 AT2 null lungs and to progress toward the center of the lung (FIG. 5C).

Together, these results indicate that the loss of Cdc42 in AT2 cells leads to progressive lung fibrosis in PNX-treated mice. Moreover, this progressive lung fibrosis phenotype also occurs in no-PNX-treated Cdc42 AT2 null mice starting from around 12 months of age.

Fibroblastic foci are considered a relevant morphologic marker of progressive pulmonary fibrosis and are recognized as sites where fibrotic responses are initiated and/or perpetuated in progressive pulmonary fibrosis³¹. The fibroblastic foci contain proliferating α- SMA⁺fibroblasts³². So, interested in characterizing the proliferation of the various stromal cell types in fibrotic lungs, we stained the lungs of Cdc42 AT2 null mice with antibodies against α- SMA as well as the cell proliferation marker Ki67 (FIG. 6A). Some α-SMA⁺fibroblasts started to accumulate next to a cluster of AT2 cells in the relative normal alveolar regions of Cdc42 AT2 null lungs (area 1, FIG. 6A). And the dense fibrosis region of the lungs is filled with α-SMA⁺fibroblasts (area 2, FIG. 6A). This analysis revealed that all of the fibrotic lungs contained proliferating α-SMA⁺fibroblasts (FIGS. 6A and 6B), indicating that these mouse stromal cells contribute to the development of lung fibrosis. **P<0.01, Student's t test.

All these results demonstrate that deletion of Cdc42 in AT2 cells leads to IPF like progressive pulmonary fibrosis in mice, and therefore, a mouse model of IPF like progressive lung fibrosis is established and can be used to study human IPF disease.

5. Discussion

As shown above, the loss of Cdc42 in AT2 cells leads to progressive lung fibrosis following lung injury. The progressive development of lung fibrosis that we observed here is apparently similar to the pathological process that occurs in IPF patients, in which fibrosis initially starts at peripheral regions of the lung before slowly proceeding inwards, eventually affecting entire lung lobes.

All these results demonstrate that deletion of Cdc42 in AT2 cells leads to IPF like progressive pulmonary fibrosis in mice, and therefore, a mouse model of IPF like progressive lung fibrosis is established and can be used to study human IPF disease.

### REFERNCES

1Wynn, T. A. Cellular and molecular mechanisms of fibrosis. The Journal of pathology 214, 199-210, doi:10.1002/path.2277 (2008).
2Wynn, T. A. & Ramalingam, T. R. Mechanisms of fibrosis: therapeutic translation for fibrotic disease. Nature medicine 18, 1028-1040, doi:10.1038/nm.2807 (2012).
3Mehal, W. Z., Iredale, J. & Friedman, S. L. Scraping fibrosis: expressway to the core of fibrosis. Nature medicine 17, 552-553, doi:10.1038/nm0511-552 (2011).
4Barkauskas, C. E. & Noble, P. W. Cellular mechanisms of tissue fibrosis. 7. New insights into the cellular mechanisms of pulmonary fibrosis. American journal of physiology. Cell physiology 306, C987-996, doi:10.1152/ajpce11.00321.2013 (2014).
5Rock, J. R. et al. Multiple stromal populations contribute to pulmonary fibrosis without evidence for epithelial to mesenchymal transition. Proceedings of the National Academy of Sciences of the United States of America 108, E1475-1483, doi:10.1073/pnas.1117988108 (2011).
6Gross, T. J. & Hunninghake, G. W. Idiopathic pulmonary fibrosis. New England Journal of Medicine 345, 517-525 (2001).
7Vyalov, S. L., Gabbiani, G. & Kapanci, Y. Rat alveolar myofibroblasts acquire alpha- smooth muscle actin expression during bleomycin-induced pulmonary fibrosis. The American journal of pathology 143, 1754 (1993).
8King Jr, T. E., Pardo, A. & Selman, M. Idiopathic pulmonary fibrosis. The Lancet 378, 1949-1961 (2011).
9Plantier, L. et al. Ectopic respiratory epithelial cell differentiation in bronchiolised distal airspaces in idiopathic pulmonary fibrosis. Thorax 66, 651-657, doi:10.1136/thx.2010.151555 (2011).
10Steele, M. P. & Schwartz, D. A. Molecular mechanisms in progressive idiopathic pulmonary fibrosis. Annual review of medicine 64, 265-276, doi:10.1146/annurev-med- 042711-142004 (2013).
11Camelo, A., Dunmore, R., Sleeman, M. A. & Clarke, D. L. The epithelium in idiopathic pulmonary fibrosis: breaking the barrier. Frontiers in pharmacology 4, 173, doi:10.3389/fphar.2013.00173 (2014).
12Barkauskas, C. E. et al. Type 2 alveolar cells are stem cells in adult lung. The Journal of clinical investigation 123, 3025-3036, doi:10.1172/JCI68782 (2013).
13Desai, T. J., Brownfield, D. G. & Krasnow, M. A. Alveolar progenitor and stem cells in lung development, renewal and cancer. Nature 507, 190-194, doi:10.1038/nature12930 (2014).
14Haies, D. M., Gil, J. & Weibel, E. R. Morphometric study of rat lung cells: I. Numerical and dimensional characteristics of parenchymal cell population. American Review of Respiratory Disease 123, 533-541 (1981).
15Selman, M. & Pardo, A. Idiopathic pulmonary fibrosis: an epithelial/fibroblastic cross- talk disorder. Respiratory research 3, 3 (2001).
16Kropski, J. A., Blackwell, T. S. & Loyd, J. E. The genetic basis of idiopathic pulmonary fibrosis. European Respiratory Journal 45, 1717-1727 (2015).
17 Goodwin, A. T. & Jenkins, G. Molecular endotyping of pulmonary fibrosis. Chest 149, 228-237 (2016).
18Xu, Y. et al. Single-cell RNA sequencing identifies diverse roles of epithelial cells in idiopathic pulmonary fibrosis. JCI insight 1 (2016).
19Meltzer, E. B. & Noble, P. W. Idiopathic pulmonary fibrosis. Orphanet journal of rare diseases 3, 8, doi:10.1186/1750-1172-3-8 (2008).
20Richeldi, L., Collard, H. R. & Jones, M. G. Idiopathic pulmonary fibrosis. The Lancet 389, 1941-1952, doi:10.1016/s0140-6736(17)30866-8 (2017).
21TE, J. K. et al. Idiopathic pulmonary fibrosis. American journal of respiratory and critical care medicine 161, 646-664 (2000).
22Lynch, D. A. et al. High-resolution computed tomography in idiopathic pulmonary fibrosis: diagnosis and prognosis. American journal of respiratory and critical care medicine 172, 488-493 (2005).
23Noble, P. W. et al. Pirfenidone in patients with idiopathic pulmonary fibrosis (CAPACITY): two randomised trials. The Lancet 377, 1760-1769 (2011).
24Raghu, G. et al. An official ATS/ERS/JRS/ALAT statement: idiopathic pulmonary fibrosis: evidence-based guidelines for diagnosis and management. American journal of respiratory and critical care medicine 183, 788-824 (2011).
25Chen, L. et al. Cdc42 deficiency causes Sonic hedgehog-independent holoprosencephaly. Proceedings of the National Academy of Sciences 103, 16520-16525 (2006).
26Leveen, P. et al. Induced disruption of the transforming growth factor beta type II receptor gene in mice causes a lethal inflammatory disorder that is transplantable. Blood 100, 560-568 (2002).
27Council, N. R. Guide for the care and use of laboratory animals. (National Academies Press, 2010).
28Foltz, C. J. & Ullman-Cullere, M. Guidelines for assessing the health and condition of mice. Resource 28 (1999).
29Wang, Y. et al. Pulmonary alveolar type I cell population consists of two distinct subtypes that differ in cell fate. Proceedings of the National Academy of Sciences, 201719474 (2018).
30Liu, Z. et al. MAPK-Mediated YAP Activation Controls Mechanical-Tension-Induced

Pulmonary Alveolar Regeneration. Cell reports 16, 1810-1819, doi:10.1016/j.celrep.2016.07.020 (2016).
31Lynch, D. A. et al. Diagnostic criteria for idiopathic pulmonary fibrosis: a Fleischner Society White Paper. The Lancet Respiratory Medicine 6, 138-153, doi:10.1016/s2213- 2600(17)30433-2 (2018).
32Lederer, D. J. & Martinez, F. J. Idiopathic Pulmonary Fibrosis. The New England journal of medicine 378, 1811-1823, doi:10.1056/NEJMra1705751 (2018).

1. A method for constructing an animal model of pulmonary fibrosis, comprising a step of increasing mechanical tension on alveolar epithelium of an animal.

2. The method of claim 1, wherein before the step of increasing the mechanical tension on the alveolar epithelium, the animal undergoes a pneumonectomy (PNX).

3. The method of claim 1, wherein the step of increasing the mechanical tension on the alveolar epithelium includes a step of increasing mechanical tension on alveolar type II (AT2) cells.

4. The method of claim 3 wherein the step of increasing the mechanical tension on alveolar type II (AT2) cells involves a step of deactivating Cdc42 in AT2cells.

5. The method of claim 4, wherein deactivating Cdc42 in AT2 cells involves deleting, disrupting, inserting, knocking-out or inactivating Cdc42 genes in AT2 cells.

6. The method of claim 1, comprising a step of knocking-out Cdc42 gene in AT2 cells in a PNX-treated animal.

7. The method of claim 6, wherein the knockout of Cdc42 gene in AT2cells leads to progressive lung fibrosis in the PNX-treated animal.

8. The method of claim 7, wherein the progressive lung fibrosis phenotype occurs in non-PNX-treated Cdc42 AT2 null animals in middle age and old age.

9. The method of claim 6, wherein in the lungs of Cdc42 AT2 null animals, fibroblastic foci are developed.

10. The method of claim 1, wherein the animal is mouse, rabbit, rat, canine, pig, horse, cow, sheep, monkey or chimpanzee.

11. An animal model of pulmonary fibrosis constructed through increasing mechanical tension on alveolar epithelium of an animal.

12. The animal model of claim 11, wherein the animal model is constructed through increasing mechanical tension on AT2cells of the animal, and/or the mechanical tension on the alveolar epithelium of the animal is increased.

13. (canceled)

14. The animal model of claim 12, wherein Cdc42 gene in AT2 cells is deactivated, deleted, disrupted, inserted, knocked-out or inactivated.

15-16. (canceled)

17. The animal model of claim 11, wherein the animal model shows progressive lung fibrosis phenotype after undergoing PNX and/or develops fibrotic changes after the pneumonectomy (PNX) treatment.

18. The animal model of claim 11, wherein the animal model without undergoing PNX shows progressive lung fibrosis phenotype in middle age and old age.

19. The animal model of claim 11, wherein the fibroblastic foci are developed.

20.(canceled)

21. The animal model of claim 14, wherein the animal model shows genotype of Cdc42 AT2 null.

22. The animal model of claim 14, wherein the animal model is Cdc42 AT2 null mouse.

23. The animal model of claim 11, wherein the animal is mouse, rabbit, rat, canine, pig, horse, cow, sheep, monkey, or chimpanzee.

24-32. (canceled)

33. A method for screening candidate drugs or a drug target for treating pulmonary fibrosis of animals and human beings, comprising using an animal model of pulmonary fibrosis constructed through increasing mechanical tension on alveolar epithelium of an animal, or using an AT2cell of lung in which the mechanical tension on the alveolar epithelium is increased.

34. (canceled)

35. The mcthod of claim 33, wherein one kind of drug target, involving a positive feedback loop of TGFβ/SMAD signaling in human or mouse AT2cells is searched out.

36. The method of claim 35, wherein the autocrine TGFβin human or mouse AT2 cells activates TGFβ/SMAD signaling in AT2 cells.

37. The method of claim 35, wherein the expression level of autocrine TGFβin both human and mouse AT2 cells is significantly increased by mechanical stretching.

38. The method of claim 37, wherein the positive feedback loop of TGFβ/SMAD signaling in stretched human and mouse AT2 cells further results in the increased expression level of autocrine TGFβ.

39. (canceled)

40. The method of claim 33, wherein the animal is mouse, rabbit, rat, canine, pig, horse, cow, sheep, monkey or chimpanzee.

41. A method for evaluation of the therapeutic effects or prognosis evaluation of pulmonary fibrosis using the animal model of claim 11.

42. (canceled)

43. A method for detecting the animal model of claim 11 using a pair of primers designed on the basis of the sequences shown by SEQ ID NO:4.

44. The method of claim 43, wherein the primers for detecting the said animal model are shown as followed:
Forward:
(SEQ ID NO: 1)
CTGCCAACCATGACAACCTAA;
Reverse:
(SEQ ID NO: 2)
AGACAAAACAACAAGGTCCAG.

45. The method of claim 1, wherein the pulmonary fibrosis is idiopathic pulmonary fibrosis (IPF).

46. The animal model of claim 11, wherein the pulmonary fibrosis is idiopathic pulmonary fibrosis (IPF).

47. The method of claim 33, wherein the pulmonary fibrosis is idiopathic pulmonary fibrosis (IPF).

48. The method of claim 6, wherein the step of knocking-out Cdc42 gene in AT2 cells in the PNX-treated animal comprises knocking out Cdc42 specifically in lung AT2 cells by using a Spc-CreER allele.
